(19) United States
(12) Patent Application Publication
(10) Pub. No.: US 2018/0002650 A1
(43) Pub. Date: Jan. 4, 2018

(54) SHAKER CAPABLE OF BEING USED IN CARBON DIOXIDE INCUBATOR AND CELL SUSPENSION CULTURE METHOD

(71) Applicant: ZHENGZHOU VIRI BIOTECH CO.,LTD., Zhengzhou (CN)

(72) Inventor: Zhiqiang Han, Zhengzhou (CN)

(21) Appl. No.: 15/201,435

(22) Filed: Jul. 2, 2016

(51) Int. Cl. C12M 3/06; C12M 1/36; C12M 1/34; C12N 5/071; C12M 1/00

(52) U.S. Cl. CPC C12M 27/16; C12N 5/0602; C12M 41/14; C12M 41/34; C12M 41/48

(57) ABSTRACT

The present invention relates to a shaker capable of being used in a carbon dioxide incubator and a cell suspension culture method. The shaker comprises a power supply system, a driving device and an executing mechanism. The power supply system is connected with the driving device through a power line, of which the length can pass through the inside and the outside of the incubator through a joint position of an inner door of the incubator and a sealing gasket on a frame of an incubator door or through a through hole in the back of the incubator. The power line enables the driving device and the executing mechanism, and the power supply system to be arranged inside and the outside the incubator respectively, thereby solving problems of temperature-control failure of the incubator resulting from excessive accumulated heat produced by the power supply system and damage to electronic elements of the incubator caused by dampness in the incubator when a conventional shaker is used in the carbon dioxide incubator.

### TECHNICAL FIELD

The present invention relates to the field of biological culture auxiliary equipment, and in particular to a shaker capable of being used in a carbon dioxide incubator and a cell suspension culture method.

### BACKGROUND

A carbon dioxide (CO2) incubator is a device for simulating the growing environment of similar cells/tissues in vivo so as to perform vitro cell and tissue culture and culture of some special microorganisms, and the carbon dioxide incubator is widely applied to the research fields such as cellular kinetic study, mammal cell secretion collection, carcinogenic or toxicological effect of various physical and chemical factors, antigen research and production, culture of hybridoma cells for producing antibodies, in vitro fertilization (IVF), stem cell, immune cell, tissue engineering, drug screening, etc.

The carbon dioxide (CO2) incubator is a common cell culture device for static cultivation of tissue cells through containers such as a petri dish, a culture bottle, a culture tank and a culture bag, and the cell growth environment simulated by the carbon dioxide (CO2) incubator comprises stable temperature (usually 37 degrees centigrade), precise carbon dioxide level (usually 5%), constant PH (the PH is usually 7.2-7.4), high relative saturated humidity (usually 95%), etc. If an ordinary carbon dioxide (CO2) incubator is utilized to perform suspension cell culture, the shaker needs to be placed in the carbon dioxide (CO2) incubator, and suspension culture is performed on cells through the shaker. However, due to the fact that in an power supply system of an ordinary shaker, electronic elements such as a voltage regulator, a transformer and a rectifier are heat-producing elements and elements which are short-circuited and damaged easily when subject to dampness, when an ordinary shaker is used in the carbon dioxide (CO2) incubator, temperature-control failure (that is, the actual temperature inside the incubator exceeds a set temperature) of the incubator occurs due to excessive heat produced by electronic elements and elements easily damaged due to dampness are short-circuited and burned down. Moreover, a regulating system and a display system of the ordinary shaker are integrated with a host; when the ordinary shaker is placed in the incubator, an incubator door must be opened so as to observe the rotating speed and carry out on-off regulation in case of need; however, it has an adverse effect on incubator temperature and carbon dioxide (CO2) concentration when the incubator door is opened for observation and regulation. Although a special carbon dioxide (CO2) incubator shaker integrating a special carbon dioxide (CO2) incubator and a shaker is available, the shaker not only is extremely expensive in cost, but also is mechanically fixed in the incubator, so a shaking mode, such as usual plane rotation movement, cannot be changed randomly, and the shaking mode cannot be changed randomly into a three-dimensional (3D) space rotation or seesaw type swing movement mode. Besides, what is more important, the number of presently used ordinary carbon dioxide incubators is large, and the costs of enterprises or research institutes are obviously increased if the high-cost special carbon dioxide (CO2) incubator shaker is specially purchased only for cell suspension culture. However, if the shaker structure can be implemented through some means, so that the shaker can be placed in the carbon dioxide (CO2) incubator to be used, the aforementioned suspension cell culture problem can be solved through the ordinary carbon dioxide (CO2) incubator.

Seen from the aspect of structural features, the carbon dioxide (CO2) incubator has good heat insulation and sealing performance, an elastic sealing gasket is subject to press fitting at the joint position of the inner door of the incubator and the frame of the incubator door, and when the carbon dioxide (CO2) incubator door is closed, the sealing gasket has a sealing effect so as to prevent instability of carbon dioxide (CO2) concentration caused by gas leakage. Moreover, the backs of most of incubators are provided with through holes; the through holes are usually plugged through corresponding rubber plugs, are in a normally closed state and are unplugged only under specific conditions, and thus the joint position of the inner door of the incubator and the sealing gasket on the frame of the incubator door or the through hole in the back of the incubator serves as the ideal passing path for a power line and a signal line which go in and out of the incubator. Since the ordinary carbon dioxide (CO2) incubator only comprises a heating device and does not contain a refrigerating device and the carbon dioxide (CO2) incubator is usually used at room temperature (at about 26 degrees centigrade) or in the environment at a temperature lower than the set usual culture temperature of 37 degrees centigrade, the accurate temperature regulation can be achieved by regulating the heating device, and thus a refrigerating device is not needed. If the environment temperature is higher than the set culture temperature, only when the environment temperature is regulated through an air conditioner to be lower than the set temperature can the incubator be used. Although the requirements on internal and external temperature changes of any carbon dioxide (CO2) incubator can be met by adding the refrigerating device, the structure cost of the carbon dioxide (CO2) incubator and complexity in precise regulation are increased, and thus almost all carbon dioxide (CO2) incubators are provided with no refrigerating device.

The structure of the existing shaker is embodied to some extent in the specification of the Chinese patent with a publication number of CN203648452U, and the shaker mainly comprises a power supply system, a driving device and a mechanical shaking device, namely an executing mechanism. The power supply system mainly comprises a voltage stabilizing transformer and a rectifier. The driving device is mostly a motor, and the executing mechanism is a plane rotation mechanism, a space rotation mechanism, or a seesaw type swing mechanism, etc. When the shaker is used, the power supply system introduces electric energy with an appropriate parameter for the driving device, the driving device performs action and drives the executing mechanism to move, and the action of the executing mechanism finally becomes the finally needed shaking movement of the shaker. In order to meet different requirements, some shakers comprise electronic regulating systems. The electronic regulating system mainly comprises a sensor, a signal line, a speed regulator and a display. The running speed of the driving device can be controlled through the electronic regulating system, so as to control the shaking speed of the executing mechanism. However, the same as what is described in the specification of the Chinese patent with a publication number of CN203648452U, in the existing shaker, the power supply system, the driving device and the electronic regulating system are arranged to be an integrated structure through the arrangement of a base.

Through the above analysis of the ordinary carbon dioxide (CO2) incubator and the structure of the existing shaker, we can know that problems in two aspects are faced mainly when the ordinary shaker is used in the carbon dioxide (CO2) incubator, on one hand, the quantity of heat produced by electronic elements of the power supply system and an electronic speed regulating system is large, an incubator body of the carbon dioxide (CO2) incubator has good heat insulation property, and heat accumulation may lead to overheating in the carbon dioxide (CO2) incubator and temperature control failure; on the other hand, the environment humidity in the carbon dioxide (CO2) incubator is high; as mentioned above, the internal environment humidity of the carbon dioxide (CO2) incubator is usually 95%, and the damp environment may lead to the result that the electronic elements of the power supply system and the electronic speed regulating system are subject to dampness and are short-circuited and burned down.

### SUMMARY

An objective of the invention is to provide a shaker capable of being used in a carbon dioxide incubator, so as to solve the problems that when placed in a carbon dioxide (CO2) incubator, an ordinary shaker accumulates heat and is damaged due to dampness. The objective of the invention is further to provide a cell suspension culture method performed by the shaker capable of being used in the carbon dioxide (CO2) incubator.

In order to solve the aforementioned problems, the shaker capable of being used in the carbon dioxide incubator according to the invention is in the following technical solution: the shaker capable of being used in the carbon dioxide incubator comprises a power supply system, a driving device and an executing mechanism; the power supply system is connected with the driving device through a power line, of which the length can pass through the inside and the outside of the incubator through a joint position of an inner door of the incubator and a sealing gasket on a frame of an incubator door or through a through hole in the back of the incubator; and the power line enables the driving device and the executing mechanism, and the power supply system to be arranged inside and the outside of the incubator respectively.

The power supply system comprises a voltage regulator and a rectifier.

The driving device is a damp-proof motor.

The executing mechanism is one of a plane rotation mechanism, a space rotation mechanism and a seesaw type swing mechanism.

The shaker further comprises an electronic speed regulating system, and the electronic speed regulating system comprises an electronic speed regulator, a display and a rotating speed sensor. The rotating speed sensor is arranged on the driving device. The electronic speed regulator and the display are connected with the sensor through a signal line, and the signal line enables the rotating speed sensor, and the electronic speed regulator and the display to be arranged inside and outside the carbon dioxide incubator respectively.

The power supply system, the electronic speed regulator and the display are integrally installed in one control box.

The power line and the signal line are formed by different branches of a flat cable integrated together, and the flat cable is connected with the power supply system, the electronic speed regulator and the display in the control box directly or through a flat cable connecting plug.

A cell suspension culture method is in the following technical solution: according to the cell suspension culture method, the driving device and the executing mechanism, and the power supply system of the shaker are arranged inside and outside the ordinary carbon dioxide incubator respectively, and the power supply system is connected with the driving device through the power line; the power line penetrates the incubator through the joint position of the inner door of the incubator and the sealing gasket on the frame of the incubator door or through the through hole in the back of the incubator, a culture container containing culture cells is placed on the executing mechanism of the shaker, and the suspension culture can be performed by starting the shaker.

One end of the signal line passing through the speed regulating system is connected with the sensor placed on the driving device in the incubator, the other end of the signal line is connected with the speed regulator and the display which are placed outside the incubator, and the path for the signal line to penetrate the carbon dioxide incubator is the same as that of the power line.

The power supply system and the driving device of the shaker capable of being used in the carbon dioxide incubator according to the invention are connected through a power line, and the power line enables the driving device and the executing mechanism, and the power sup ply system to be arranged inside and outside the carbon dioxide (CO2) incubator respectively; thus, in use, the main heat-producing and easily-damped parts of the shaker are moved out of the carbon dioxide (CO2) incubator, and accordingly the problems of heat accumulation and damage to the shaker occurring when the shaker is used in the ordinary carbon dioxide (CO2) incubator are solved. Due to the fact that the influencing factor on incubator temperature caused by heat produced by electronic elements of the power supply system and a regulating system is removed, the power of the motor is allowed to be relatively increased.

### BRIEF DESCRIPTION OF DRAWINGS

FIG. 1 is a structural schematic diagram of Embodiment 1 of a shaker capable of being used in a carbon dioxide incubator;

FIG. 2 is another structural schematic diagram (displaying a disconnection state of a flat cable connecting plug) of Embodiment 1 a shaker capable of being used in a carbon dioxide incubator;

FIG. 3 is a structural schematic diagram (a three-dimensional rotation shaker mechanism) of an executing mechanism of Embodiment 1 of a shaker capable of being used in a carbon dioxide incubator;

FIG. 4 is a first use state external diagram (a flat cable penetrates a through hole in the back of an incubator) of Embodiment 1 of a shaker capable of being used in a carbon dioxide incubator;

FIG. 5 is a first use state internal diagram (a flat cable penetrates a through hole in the back of an incubator) of Embodiment 1 of a shaker capable of being used in a carbon dioxide incubator;

FIG. 6 is a second use state external diagram (a flat cable penetrates a joint position of an inner door of an incubator and a sealing gasket on a frame of an incubator door) of Embodiment 1 of a shaker capable of being used in a carbon dioxide incubator;

FIG. 7 is a second use state internal diagram (a flat cable penetrates a joint position of an inner door of an incubator and a sealing gasket on a frame of an incubator door) of Embodiment 1 of a shaker capable of being used in a carbon dioxide incubator;

FIG. 8 is a sectional drawing (a tray is removed from a plane rotation shaker mechanism) of Embodiment 2 of a shaker capable of being used in a carbon dioxide incubator;

FIG. 9 is an external schematic diagram (a plane rotation shaker mechanism) of Embodiment 2 of a shaker capable of being used in a carbon dioxide incubator; and

FIG. 10 is a structural schematic diagram (a seesaw shaker mechanism) of an executing mechanism of Embodiment 3 of a shaker capable of being used in a carbon dioxide incubator.

### DESCRIPTION OF EMBODIMENTS

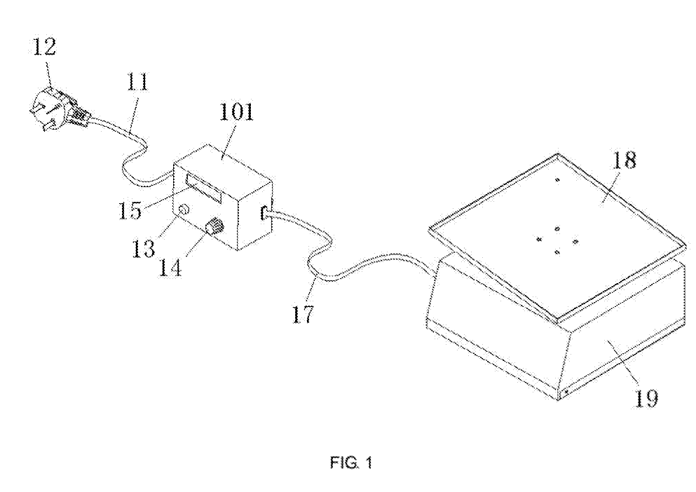

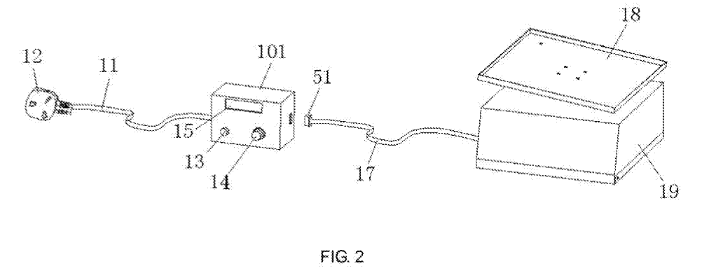

Embodiment 1 of a shaker capable of being used in a carbon dioxide incubator is as shown in FIGS. 1 to 7, wherein FIGS. 1 and 2 illustrate the main structure of the shaker. As can be seen from the figure, the shaker comprises a power supply system, an electronic speed regulating system, a driving device and an executing mechanism.

The power supply system is provided with a power supply leading-in wire 11, and one end of the power supply leading-in wire 11 for electrical connection is provided with a plug 12. In this embodiment, the power supply system comprises a voltage stabilizing transformer and a rectifier. The voltage stabilizing transformer can convert a switched-on power supply into a 36 V safe voltage, and the rectifier can rectify an alternating current into a direct current. Besides the aforementioned structure, the power supply system is further provided with a power switch 13, and the power supply can be switched on and switched off through the power switch 13. The structure of the power supply system is by means of the prior art and is not repeated here.

The electronic speed regulating system comprises an electronic speed regulator 14, a display 15 and a rotating speed sensor, wherein the rotating speed sensor is arranged on the driving device. The formation of the electronic speed regulating system is also by means of the prior art and is not repeated here.

In this embodiment, the power supply system, the electronic speed regulator and the display are integrally installed in one and the same control box 101, so as to facilitate taking and displacement. Moreover, the electronic speed regulator and the display are placed in the incubator, to facilitate observation at any time and necessary regulation; and the observation, switching on, switching off and rotating speed regulation are performed without needing to open the incubator door.

The driving device is a motor 16. In this embodiment, the motor 16 is a damp-proof motor (a waterproof motor), and heat produced in the running process can be controlled by limiting rated power of the motor 16, so as to avid the influence on the temperature in the carbon dioxide (CO2) incubator.

Though the motor of the invention can produce heat, the motor power can be selected according to the quantity of heat produced by the motor, and the motor of which the heat production efficiency is smaller than the heat dissipation efficiency of the incubator in the room temperature environment can be chosen. Although the humidity of the incubator can reach 95%, the incubator is not immersed in water after all, so the protection requirements can be met by selecting the damp-proof motor.

The power supply system is connected with the driving device through a power line, and thus the power supply system provides a working power supply for the driving device; the electronic speed regulating system is connected with the driving device through a signal line, so that the electronic speed regulating system controls the speed of the driving device. Due to the lengths of both the power line and the signal line, when the driving device of the shaker is placed in the carbon dioxide (CO2) incubator, the power supply system and the electronic speed regulator can be arranged outside the carbon dioxide (CO2) incubator, and the power supply system and the electronic speed regulating system are kept connected with the driving device. In this embodiment, the power line and the signal line are formed by different branches of a flat cable 17, and finally the power line and the signal line form the flat cable; the flat cable 17 is connected with the power supply system and the electronic speed regular in the control box through a flat cable connecting plug 51.

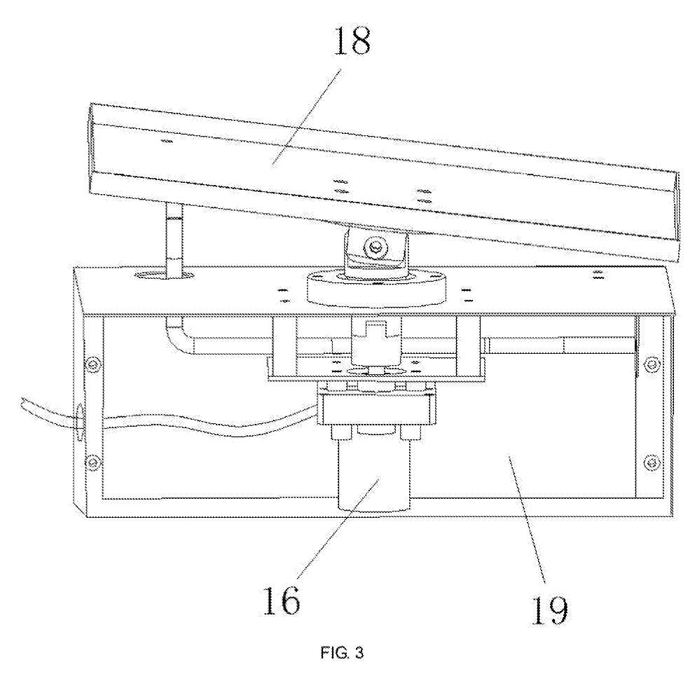

As shown in FIG. 3, the executing mechanism in this embodiment is concretely a space rotation mechanism which comprises a rotation tray 18 and a base 19. The rotation tray 18 is used for supporting a culture container. The structure of the space rotation mechanism is by means of the prior art and is not repeated here. A rotating speed sensor is assembled on the driving device; that is, in this embodiment, the motor 16 is fixedly assembled on the base 19 of the space rotation mechanism.

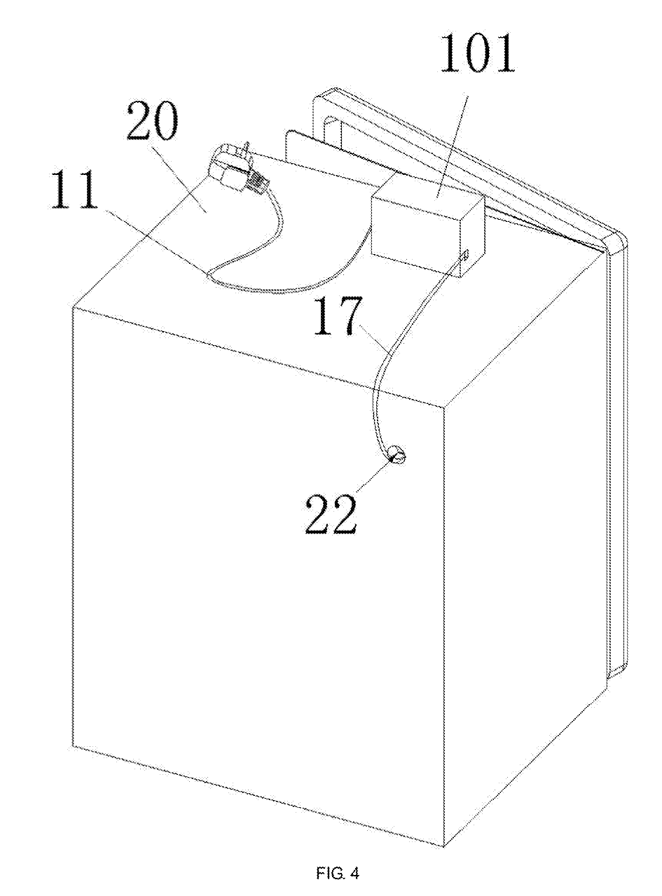

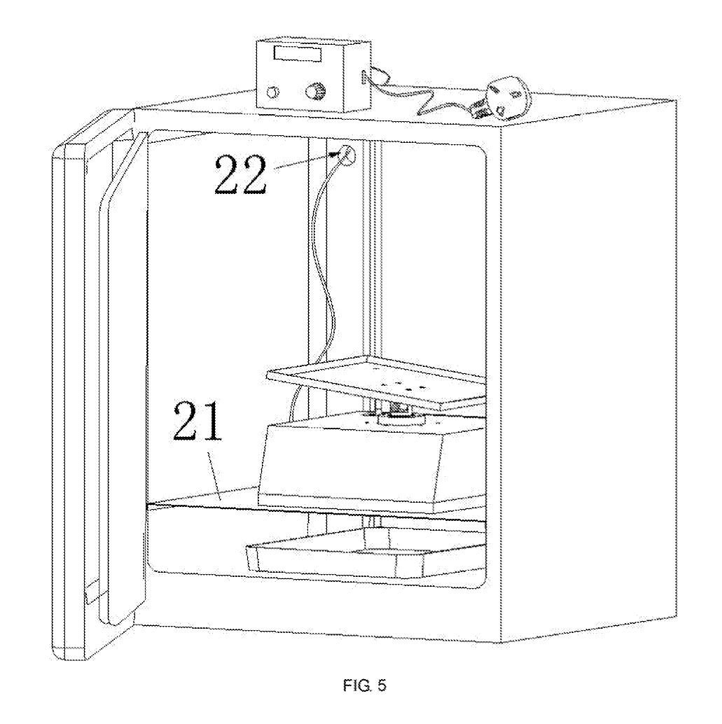

FIGS. 4 and 5 illustrate a use state of a shaker of this embodiment in an ordinary carbon dioxide (CO2) incubator. As can be seen from the figures, the driving device and the executing mechanism of the shaker are placed on a shelf 21 in an incubator body 20 of the carbon dioxide (CO2) incubator, and the power supply system, the electronic speed regulator and the display are placed outside the carbon dioxide (CO2) incubator. The power line and the signal line penetrate the inside and the outside of the incubator body 20 through the position of a through hole 22 in the back of the incubator body of the carbon dioxide (CO2) incubator. The through hole 22 in the back of the incubator body 20 of the carbon dioxide (CO2) incubator belongs to the structure (the position of the through hole is provided with a plug, and the power line and the signal line penetrate the plug to connect the inside and the outside of the carbon dioxide (CO2) incubator) of the carbon dioxide (CO2) incubator, and thus the shaker is used in the carbon dioxide (CO2) incubator under the conditions that the structure of the carbon dioxide (CO2) incubator is not damaged.

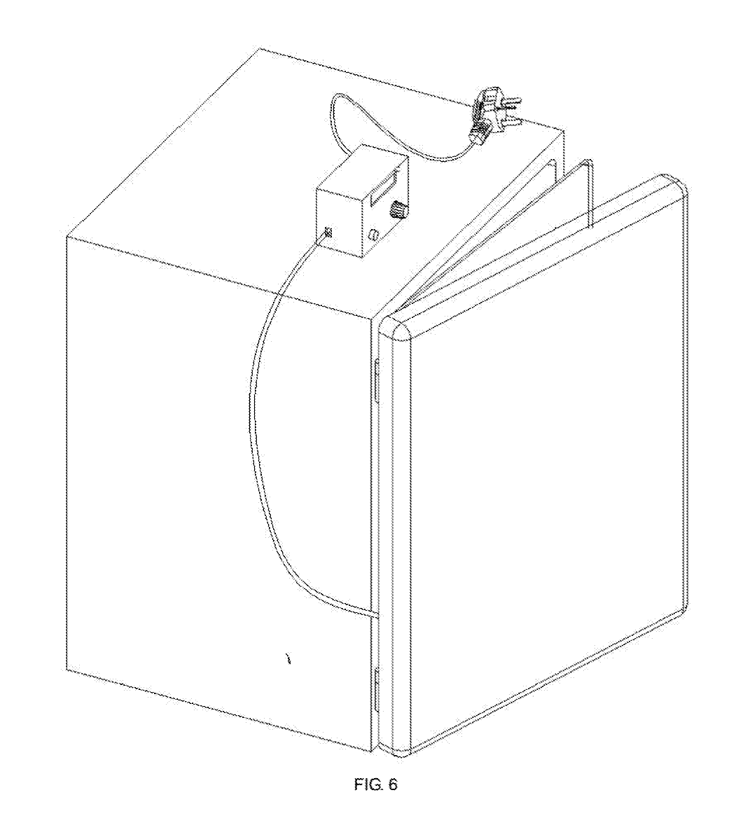

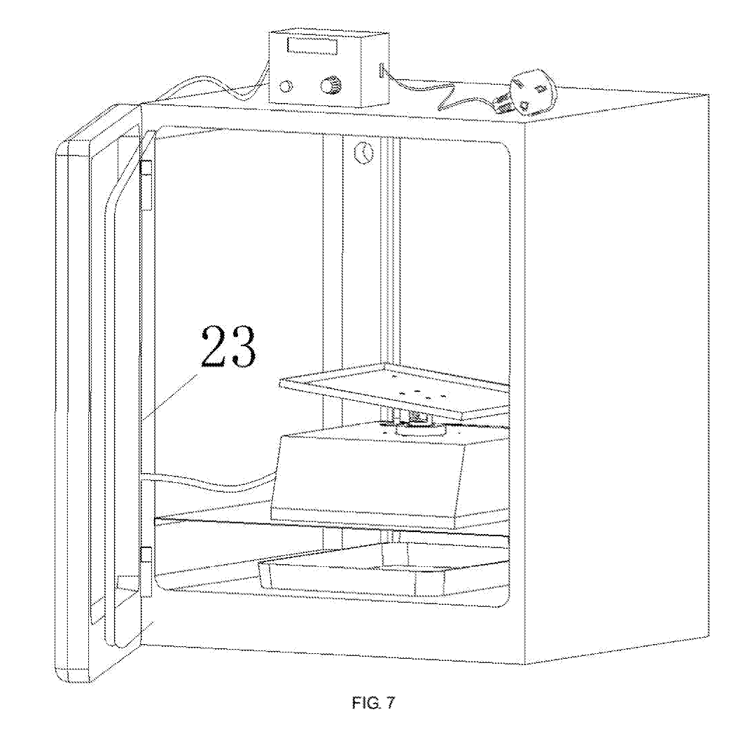

FIGS. 6 and 7 illustrate another use state of a shaker of this embodiment in an ordinary carbon dioxide (CO2) incubator. What is different from the last use state is that in this state, the power line and the signal line penetrate the inside and the outside of the incubator body through the joint position of the inner door of the carbon dioxide (CO2) incubator and the sealing gasket on the frame of the incubator door; since an elastic sealing gasket 23 is arranged at the joint position of the inner door of the carbon dioxide (CO2) incubator and the frame of the incubator door, when the power line and the signal line pass, the sealing gasket can be subject to corresponding deformation, not only are the power line and the signal line not squeezed, but also the original sealing performance of the incubator door can be maintained. It can be seen that under the use conditions, the original structure of the carbon dioxide (CO2) incubator does not need to be damaged.

Through the above introduction of the two use modes of this shaker, it can be known that the shaker can be matched with the original ordinary carbon dioxide (CO2) incubator to achieve cell suspension culture under the conditions that the structure of the original ordinary carbon dioxide (CO2) incubator is not changed. Relative to the special carbon dioxide (CO2) incubator shaker which is high in cost, the shaker is obvious more economical, and functions of the original ordinary carbon dioxide (CO2) incubator universally used on the market are brought into full play.

In other embodiments of the shaker capable of being used in the carbon dioxide incubator, the power line and the signal line can further be two independent wires; the power supply system and the electronic speed regulating system may not necessarily be integrated; since the motor does not belong to easily damaged parts in the carbon dioxide (CO2) incubator, the motor can be an ordinary motor; that is, the motor serving as the driving device may not necessarily be a damp-proof motor.

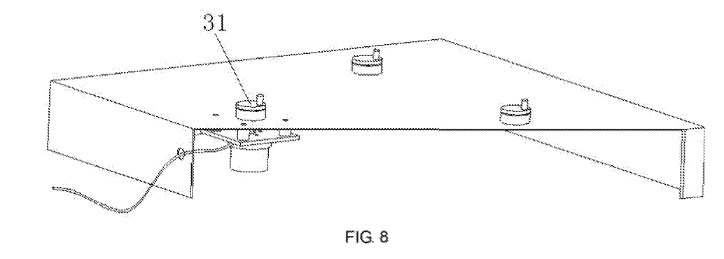

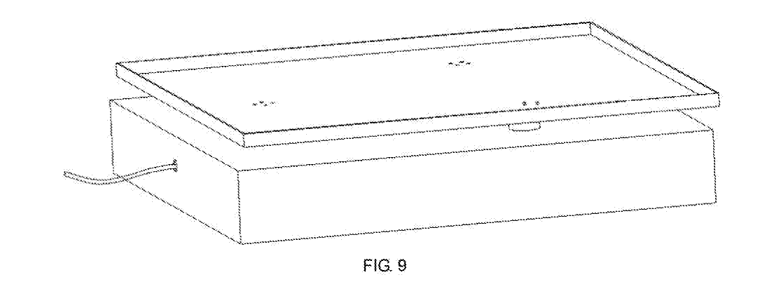

Embodiment 2 of the shaker capable of being used in the carbon dioxide incubator is as shown in FIGS. 8 and 9. This shaker capable of being used in the carbon dioxide incubator is different from Embodiment 1 of the shaker capable of being used in the carbon dioxide incubator in that in this embodiment, the executing mechanism is a plane rotation mechanism, a base of the plane rotation mechanism is connected with a tray through three eccentric shafts 31, the eccentric shafts 31 support the tray, and the tray can support a culture container and enables the three eccentric shafts 31 to move synchronously. Therefore, when the motor drives one of the three eccentric shafts 31 to rotate, the other two eccentric shafts perform following action, and thus the tray is driven by the eccentric shafts 31 to perform rotation movement in the plane.

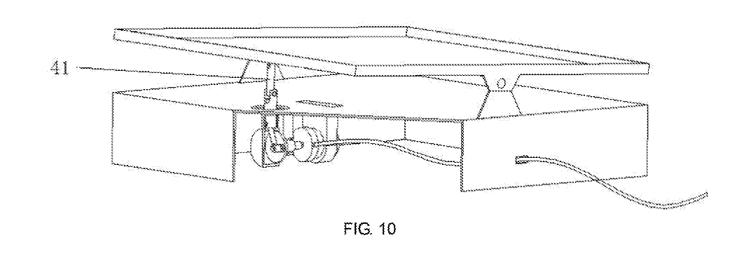

Embodiment 3 of the shaker capable of being used in the carbon dioxide incubator is as shown in FIG. 10. This shaker capable of being used in the carbon dioxide incubator is different from Embodiment 1 of the shaker capable of being used in the carbon dioxide incubator only in that in this embodiment, the executing mechanism is a seesaw type swing mechanism, and the motor drives the tray through a crankshaft and connecting rod mechanism to perform seesaw type swing.

The embodiment of the cell suspension culture method is provided. According to the method, the driving device and the executing mechanism of the shaker, and the power supply system and the electronic speed regulator are arranged inside and outside of the ordinary carbon dioxide (CO2) incubator respectively; the power supply system is connected with the driving device through the power line, the electronic speed regulating system is connected with the driving device through the signal line; the power line and the signal line penetrate the ordinary carbon dioxide (CO2) incubator through the joint position of the inner door of the carbon dioxide (CO2) incubator and the sealing gasket on the frame of the incubator door or through the through hole in the back of the carbon dioxide (CO2) incubator, cells are placed on the executing mechanism of the shaker, and the suspension cell culture can be performed by starting the shaker. The path of the power line and the path of the signal line can be the same and can also be different.

I claim:

1. A shaker capable of being used in a carbon dioxide incubator, comprising a power supply system, a driving device and an executing mechanism, wherein the power supply system is connected with the driving device through a power line, of which the length can pass through the inside and the outside of the incubator through a joint position of an inner door of the incubator and a sealing gasket on a frame of an incubator door or through a through hole in the back of the incubator; and the power line enables the driving device and the executing mechanism, and the power supply system to be arranged inside and the outside of the incubator respectively.

2. The shaker capable of being used in the carbon dioxide incubator according to claim 1, wherein the power supply system comprises a voltage regulator and a rectifier.

3. The shaker capable of being used in the carbon dioxide incubator according to claim 1, wherein the driving device is a damp-proof motor.

4. The shaker capable of being used in the carbon dioxide incubator according to claim 1, wherein the executing mechanism is one of a plane rotation mechanism, a space rotation mechanism and a seesaw type swing mechanism.

5. The shaker capable of being used in the carbon dioxide incubator according to claim 1, wherein the shaker further comprises an electronic speed regulating system, and the electronic speed regulating system comprises an electronic speed regulator, a display and a rotating speed sensor; the rotating speed sensor is arranged on the driving device; the electronic speed regulator and the display are connected with the sensor through a signal line, and the signal line enables the rotating speed sensor, and the electronic speed regulator and the display to be arranged inside and outside the carbon dioxide incubator respectively.

6. The shaker capable of being used in the carbon dioxide incubator according to claim 5, wherein the power supply system, the electronic speed regulator and the display are integrally installed in one control box.

7. The shaker capable of being used in the carbon dioxide incubator according to claim 5, wherein the power line and the signal line are formed by different branches of a flat cable integrated together, and the flat cable is connected with the power supply system, the electronic speed regulator and the display in the control box directly or through a flat cable connecting plug.

8. A cell suspension culture method, wherein the driving device and the executing mechanism, and the power supply system of the shaker are arranged inside and outside the ordinary carbon dioxide incubator respectively, and the power supply system is connected with the driving device through the power line; the power line penetrates the incubator through the joint position of the inner door of the incubator and the sealing gasket on the frame of the incubator door or through the through hole in the back of the incubator, a culture container containing culture cells is placed on the executing mechanism of the shaker, and the suspension culture can be performed by starting the shaker.

9. The cell suspension culture method according to claim 8, wherein one end of the signal line passing through the speed regulating system is connected with the sensor placed on the driving device in the incubator, the other end of the signal line is connected with the speed regulator and the display which are placed outside the incubator, and the path for the signal line to penetrate the carbon dioxide incubator is the same as that of the power line.
